(19) United States
(12) Patent Application Publication
(10) Pub. No.: US 2011/0253147 A1
(43) Pub. Date: Oct. 20, 2011

(54) BREATHING APPARATUS

(76) Inventors: Michael H. Gusky, Weston, FL (US); Judith Emaus, Vista, CA (US); Douglas Gaylord, San Diego, CA (US); Steven M. Harrington, Cardiff, CA (US)

(21) Appl. No.: 12/762,633

(22) Filed: Apr. 19, 2010

(51) Int. Cl. A61M 16/00

(52) U.S. Cl. 128/207.18

(57) ABSTRACT

A breathing apparatus includes a supply tube configured to provide a supply of air. A first and second nasal interface each include a generally spherical member having a respective projection configured to be at least partially received within a respective nasal passage of a user, and are independently movable relative to the housing. A valve is disposed between the fluid chamber and an exhaust passage for selectively allowing air to be exhausted from the fluid chamber via the exhaust passage. A diaphragm, coupled to a bias chamber, is also coupled to the valve for moving the valve between the open position and the closed position. Bias pressure within the bias chamber is regulated, at least in part, by a loading fluid impedance and a venting fluid impedance.

### TECHNICAL FIELD

The present disclosure generally relates to a breathing apparatus, and more particularly relates to a positive airway pressure-type breathing apparatus.

### BACKGROUND

Various products have been developed for the treatment of snoring and of sleep apnea. One common approach is directed at maintaining positive airway pressure of a user in an attempt to prevent the closing of the user's airways. One general variety of positive airway pressure devices is the continuous positive airway pressure (CPAP) system, which seeks to maintain a constant pressure in the user's upper airways. However, when air is provided to the user's upper airways at a constant supply pressure, normal respiration of the user may result in decreases and increases in pressure at the user's upper airways. The pressure variations caused by normal respiration, particularly increases in pressure resulting from exhalation by the user, are often found to be uncomfortable to the user.

Attempts to mitigate the problems associated with providing a constant supply pressure (and to thereby achieve constant upper airway pressure), often involve providing an air supply that may vary in pressures corresponding to the breathing cycle of the user. Specifically, such systems may reduce the pressure of the air supplied to the user during exhalation of by the user. Similarly, the systems may increase the pressure of the air supplied to the user during inhalation by the user. The decreased pressure of the air supplied during exhalation by the user may reduce the exhalation resistance experienced by the user, thereby making the use of the system somewhat more comfortable. Typically, the pressure of the air supplied to the user is controlled by controlling motor speed of a blower providing the air to the user. However, the stochastic nature of breathing, may result in substantial control system complications. Additionally, due to the pressure drop through an exhaust tube (e.g., which may exhaust the user's exhaled breath), a user may still experience uncomfortable resistance during exhalation. Attempts to reduce the exhalation resistance experienced by the user, which may result from the flow resistance through the exhaust tube, generally include providing a relatively large diameter exhaust tube between the user interface and the blower system. While the relatively large diameter tube may generally reduce the exhalation resistance experienced by the user, increasing the diameter of the tube may generally increase the stiffness of the tube making the system less comfortable for the user and increasing the likelihood that user movement will displace the user interface, thereby diminishing the benefits of the positive airway pressure system.

### SUMMARY OF THE DISCLOSURE

According to an embodiment a breathing apparatus includes a supply tube configured to provide a supply of air. A first and second nasal interface are fluidly coupled to the supply tube via a housing defining a fluid chamber. The first and second nasal interface each include a generally spherical member having a respective projection configured to be at least partially received within a respective nasal passage of a user. The first and second nasal interface are independently movable relative to the housing. A valve is disposed between the fluid chamber and an exhaust passage. The valve is moveable between a closed position, in which the valve is engaged with a valve seat, restricting air from being exhausted from the fluid chamber via the exhaust passage. The valve is also moveable to an open position, being at least partially disengaged with the valve seat, thereby allowing air to be exhausted from the fluid chamber via the exhaust passage. The valve seat includes at least one serration extending radially from a valve engagement surface. A diaphragm is coupled to the valve for moving the valve between the open position and the closed position. A bias chamber is coupled to the diaphragm for providing a bias force to the diaphragm. A loading fluid impedance couples the fluid chamber with the bias chamber for regulating the bias force based upon, at least in part, a pressure within the fluid chamber. A venting fluid impedance couples the fluid chamber with an ambient environment.

One or more of the following features may be included. The at least one serration may have a depth that increases radially away from the valve engagement surface. The breathing apparatus may further include a first seal disposed between the first nasal interface and the housing, and a second seal disposed between the second nasal interface and the housing. The at least one serration of the valve seat may include a plurality of serrations disposed about the circumference of the valve engagement surface. The exhaust passage may be configured to redirect exhaust air exiting the via the valve in a first direction to a substantially different second direction. The exhaust passage may be configured to redirect exhaust air proximate a first side of the housing to a second side of the housing generally opposed to the first side of the housing.

The loading fluid impedance may include a fluid passage having an associated loading impedance pressure drop, and the venting fluid impedance may include a fluid passage having an associated venting impedance pressure drop. The venting impedance pressure drop may be greater than the loading impedance pressure drop.

The breathing apparatus may further include an expandable member coupled to the bias chamber. The expandable member may be configured to expand in response to an increase in a bias chamber pressure associated with a deflection of the diaphragm. The breathing apparatus may further include an initial loading valve selectively fluidly coupling the fluid chamber and the bias chamber. An opening force of the valve may be based upon, at least in part, a ratio of a valve surface area and a diaphragm surface area.

According to another embodiment, a breathing apparatus includes a first supply tube configured to provide a supply of air. A first and second nasal interface are fluidly coupled to the first supply tube via a housing. The first and second nasal interface each include a generally spherical member having a respective projection configured to be at least partially received within a respective nasal passage of a user. The first and second nasal interface are independently movable relative to the housing.

One or more of the following features may be included. The breathing apparatus may further include a second supply tube configured to provide a supply of air. The second supply tube may be fluidly coupled to the first and second nasal interface via the housing. The generally spherical member of each of the first and second nasal interface may include an opening configured to provide fluid communication via the housing.

The breathing apparatus may further include a first seal disposed between the first nasal interface and the housing, and a second seal disposed between the second nasal interface and the housing. Each of the first seal and the second seal may include a brush seal. Each of the first seal and the second seal may include a felt ring. Each of the first seal and the second seal may include an o-ring.

According to yet a further embodiment, a breathing apparatus includes a supply tube configured to provide a supply of air. A first and second nasal interface are fluidly coupled to the supply tube via a housing defining a fluid chamber. The first and second nasal interface each include a generally spherical member having a respective projection configured to be at least partially received within a respective nasal passage of a user. The first and second nasal interface are independently movable relative to the housing. A valve is disposed between the fluid chamber and an exhaust passage. The valve is moveable between a closed position, restricting air from being exhausted from the fluid chamber via the exhaust passage, and an open position, allowing air to be exhausted from the fluid chamber via the exhaust passage. A diaphragm is coupled to the valve for moving the valve between the open position and the closed position. A bias chamber is coupled to the diaphragm for providing a bias force to the diaphragm. A loading fluid passage fluidly couples the bias chamber with a loading fluid source for regulating the bias force. A venting fluid impedance couples the fluid chamber with an ambient environment.

One or more of the following features may be included. The loading fluid passage may include a loading fluid impedance having an associated loading impedance pressure drop. The loading fluid impedance, fluidly coupling the fluid chamber and the bias chamber, may regulate the bias force based upon, at least in part, a pressure within the fluid chamber. The loading fluid source may include a voice coil driven source of pressurized fluid. The loading fluid source may include a blower. The loading fluid passage may fluidly couple the blower and the bias chamber. A valve may couple the loading fluid source and the bias chamber. The valve may be configured to provide a pulse width modulated duty cycle to regulate the bias force by regulating a pressure within the bias chamber.

The breathing apparatus may also include an expandable member coupled to the bias chamber. The expandable member may be configured to expand in response to an increase in a bias chamber pressure associated with a deflection of the diaphragm.

The valve may include a valve member configured to engage a valve seat in the closed position and configured to at least partially disengage the valve seat in the open position. The valve member may include a valve plate. The valve member may include a valve body having at least a first radial slot and a second radial slot. The first radial slot may be at least partially axially spaced from the second radial slot. The first radial slot and the second radial slot may be at least partially obstructed by the valve seat in the closed position.

The details of one or more implementations are set forth in the accompanying drawings and the description below. Other features and advantages will become apparent from the description, the drawings, and the claims.

### BRIEF DESCRIPTION OF THE DRAWINGS

FIG. 1 schematically depicts a breathing system including a breathing apparatus.

FIG. 2 depicts a first side view of the breathing apparatus of FIG. 1.

FIG. 3 depicts a second side view of the breathing apparatus of FIG. 1.

FIG. 4 depicts a top view of the breathing apparatus of FIG. 1.

FIG. 5 is a partial exploded view of the breathing apparatus of FIG. 1.

FIG. 6 is a partial exploded, cross-sectional view of the breathing apparatus of FIG. 1.

FIG. 7 is a cross-sectional, side view of the breathing apparatus of FIG. 1.

FIG. 8 is a cross-sectional, perspective view of the breathing apparatus of FIG. 1.

FIG. 9 is an exploded side view of the breathing apparatus of FIG. 1.

FIG. 10 depicts a first exploded perspective view of the breathing apparatus of FIG. 1.

FIG. 11 depicts a second exploded perspective view of the breathing apparatus of FIG. 1.

FIG. 12 depicts an exploded cross-sectional view of the breathing apparatus of FIG. 1.

FIG. 13 diagrammatically depicts a breathing apparatus coupled with a voice coil driven loading fluid source.

FIG. 14 diagrammatically depicts a breathing apparatus coupled with a blower loading fluid source.

FIG. 15. diagrammatically depicts a breathing apparatus including a valve controlled fluid loading.

FIG. 16 depicts a breathing apparatus including a slot valve.

FIG. 17A depicts the breathing apparatus of FIG. 16 with the slot valve in a closed position.

FIG. 17B depicts the breathing apparatus of FIG. 16 with the slot valve in an opened position.

### DETAILED DESCRIPTION OF EXEMPLARY EMBODIMENTS

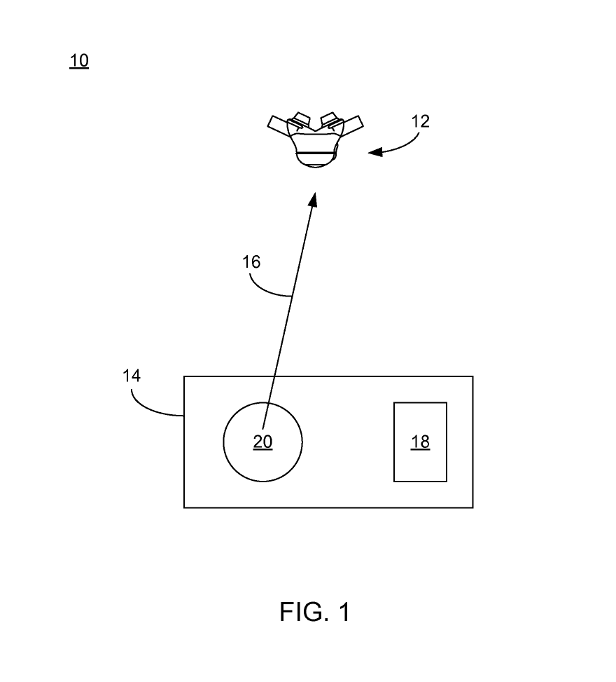

Referring to FIG. 1, breathing system 10 is generally shown including breathing apparatus 12 in conjunction with positive airway pressure (PAP) air supply 14. PAP air supply 14 may generally supply pressurized air (i.e., air at a pressure greater than ambient pressure, e.g., a pressure of 10 cm H₂O above ambient pressure, although other pressures may be equally utilized, depending upon design criteria and user need). The pressurized air generated by PAP air supply 14 may be delivered to breathing apparatus 12 via supply tube 16. In an embodiment, supply tube 16 may include a relatively small diameter (e.g., 9.5 mm inside diameter tubing) which may allow relatively un-encumbered movement of breathing apparatus 12 relative to PAP air supply 14. While a 9.5 mm inside diameter supply tube has been described above, this should not be construed as a limitation of the present disclosure as other tubing sizes may be equally utilized depending upon design criteria and user need.

As is known, PAP air supply 14 may generate varying pressure air. The pressure of the air generated may vary generally according to a breathing cycle of a user of breathing system 10. Controller 18 may detect a pressure at supply tube 16 and/or at breathing apparatus 12. For example, a relatively low pressure condition may be indicative of an inhalation by the user of breathing system 10. Similarly, a relatively high pressure condition at supply tube 16 may be indicative of an exhalation of the user of breathing system 10. Responsive to a detected inhalation by the user (e.g., in response to detecting a relatively low pressure at supply tube 16) controller 18 may cause blower 20 to spool-up, thereby increasing the pressure of the air delivered to the user (via breathing apparatus 12) via supply tube 16. In a similar manner, responsive to a detected exhalation by the user (e.g., in response to detecting relatively high pressure at supply tube 16) controller 18 may cause blower 20 to spool down, thereby decreasing the pressure of the air delivered to the user (via breathing apparatus 12) via supply tube 16.

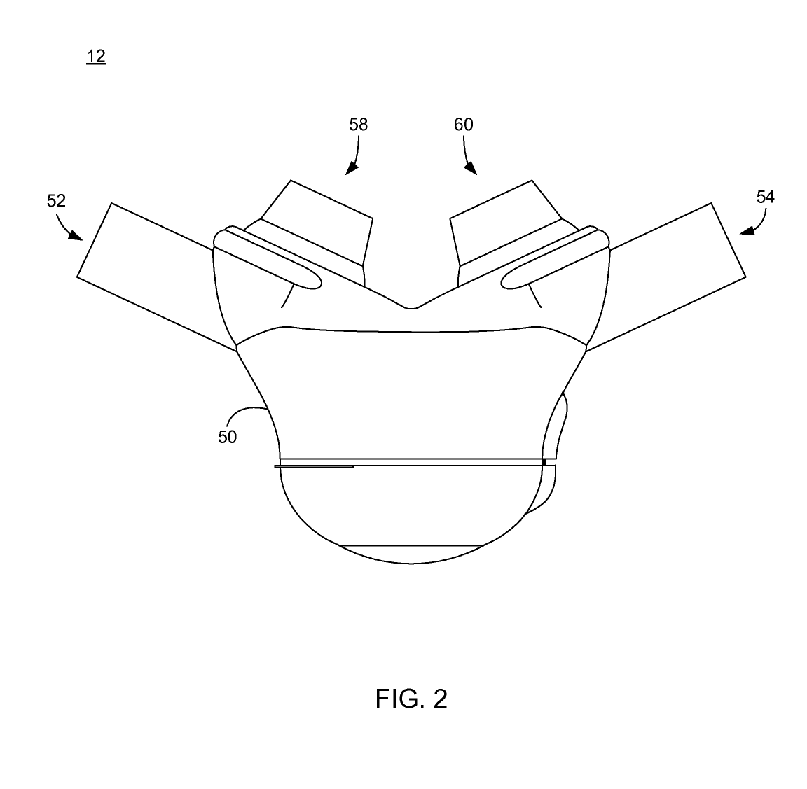

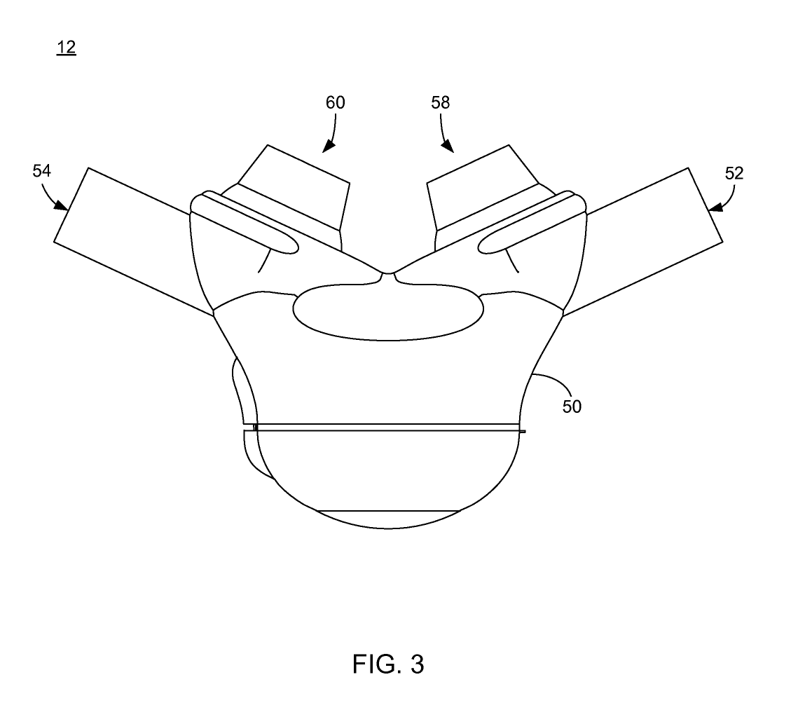

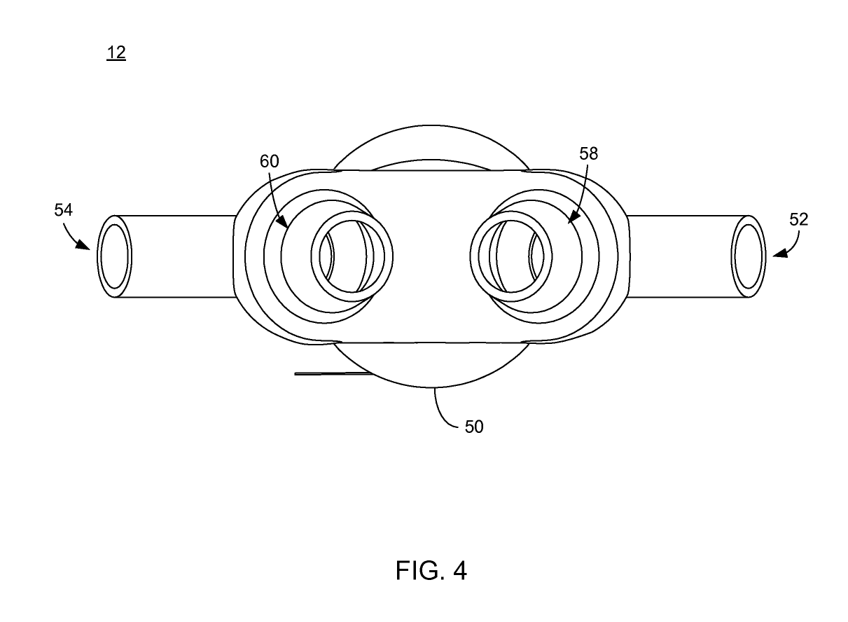

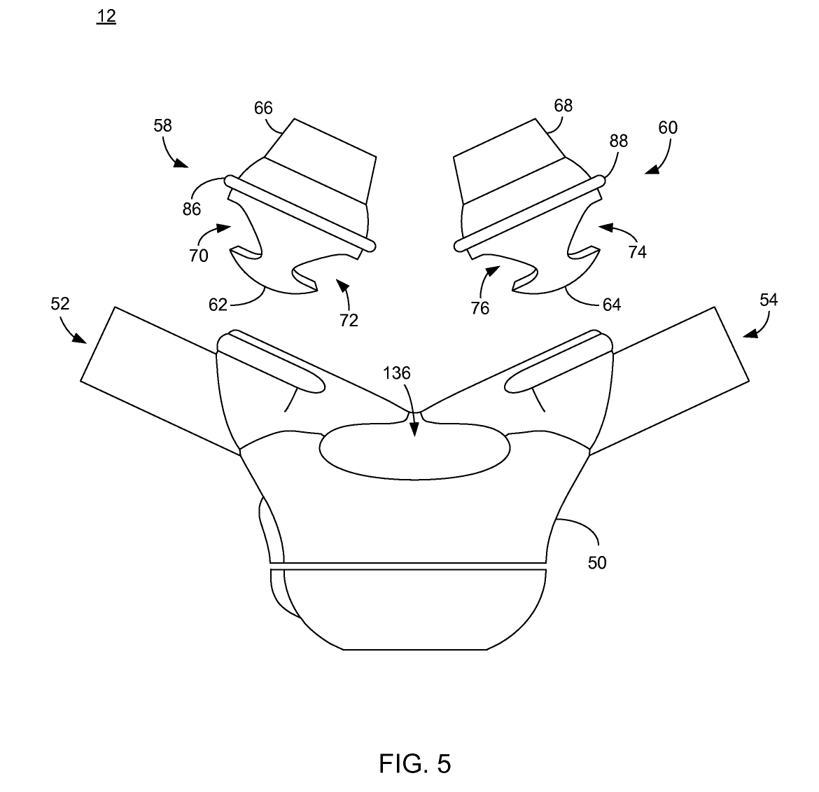

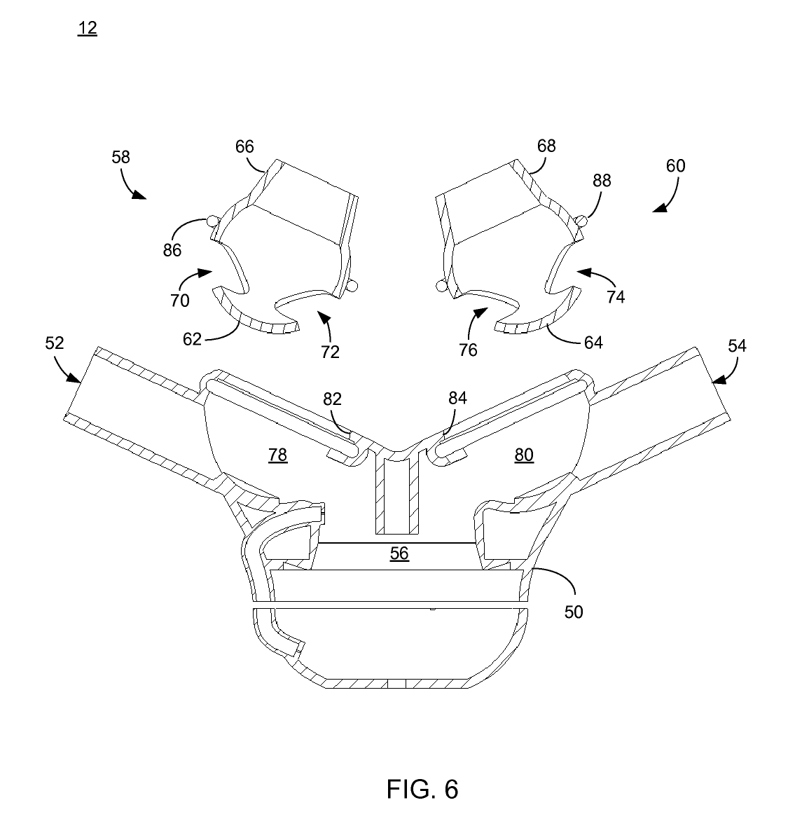

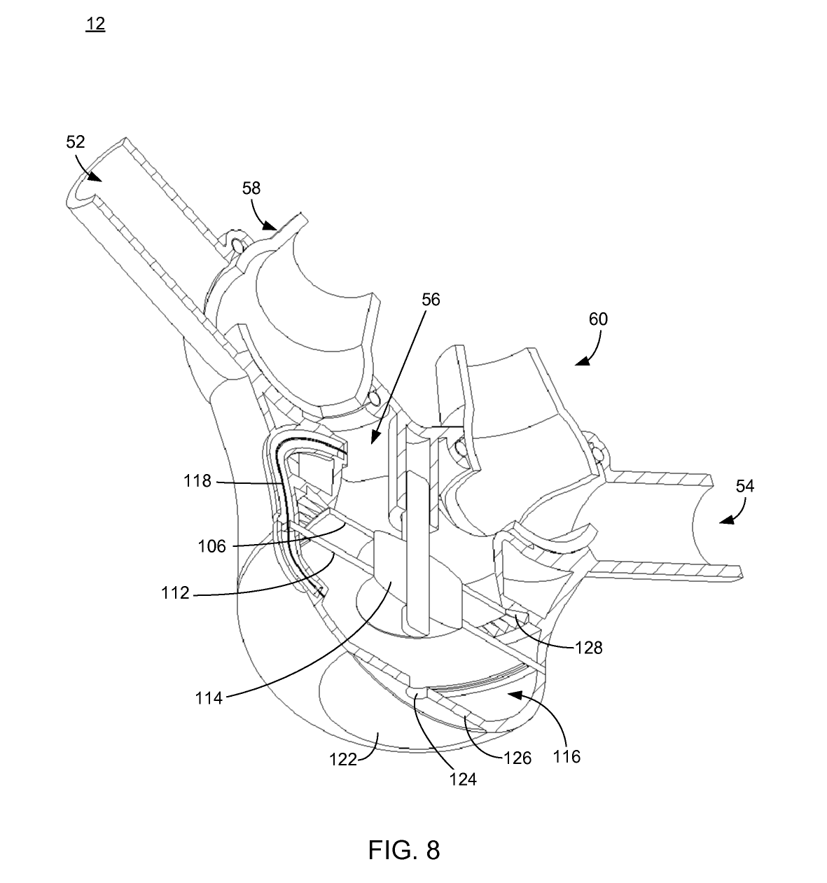

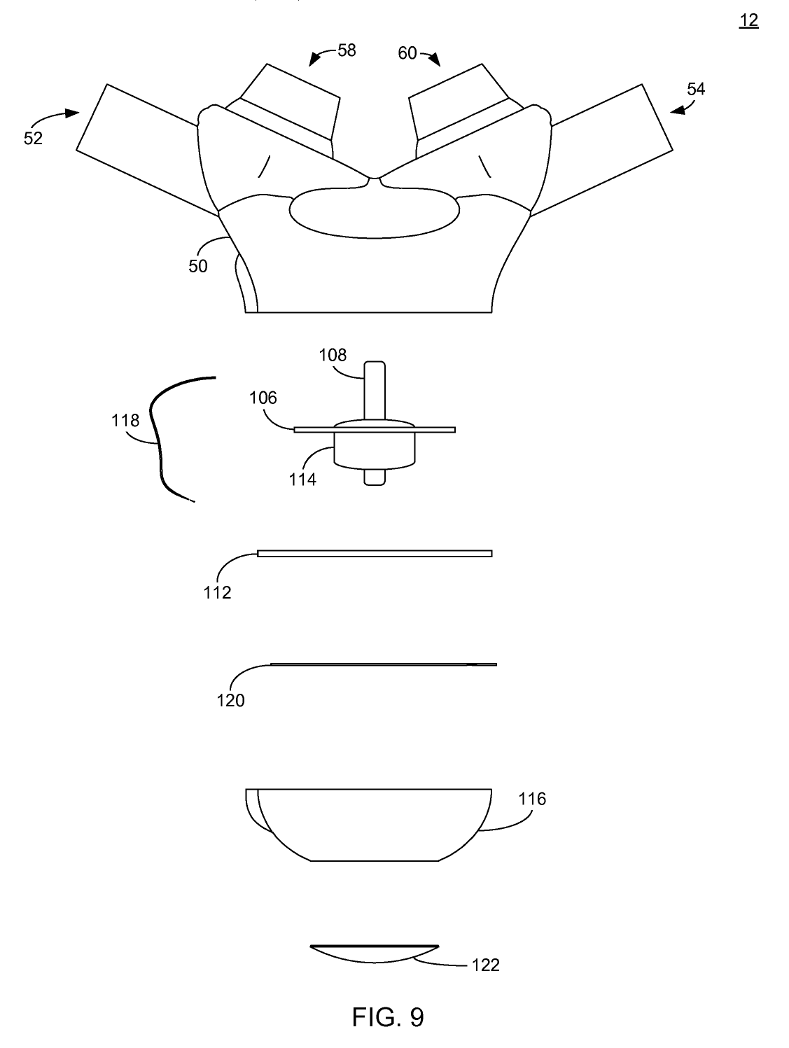

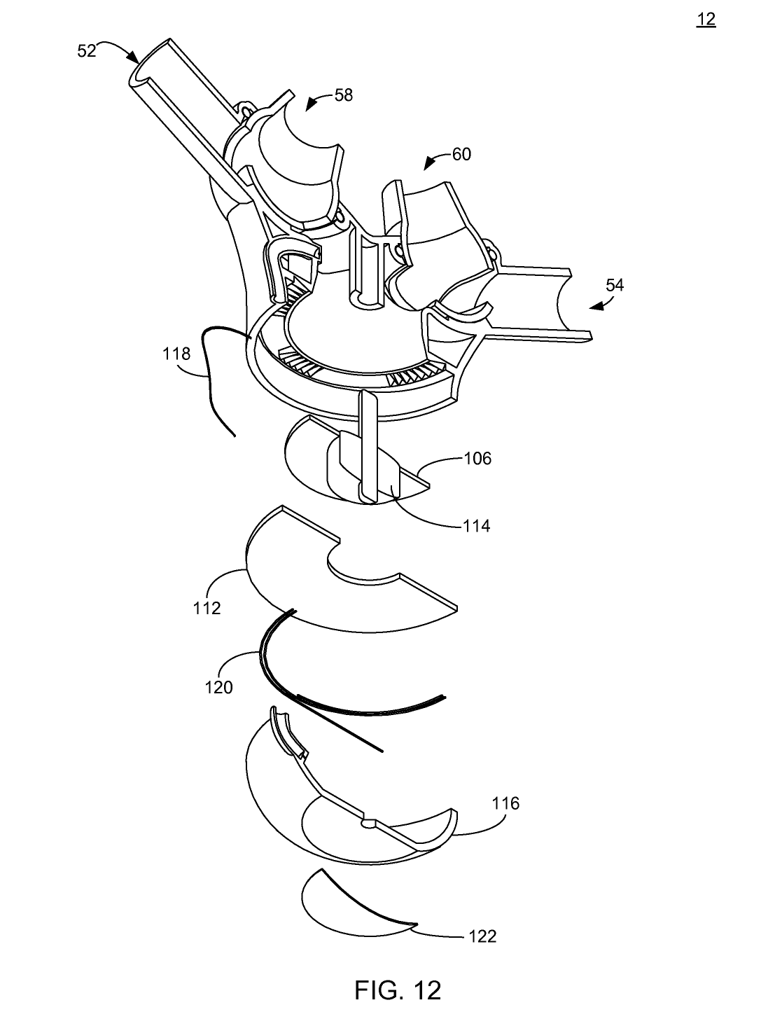

Referring to FIGS. 2 through 12, various aspects and features of breathing apparatus 12 are shown. Generally, breathing apparatus 12 may include a supply tube configured to provide a supply of air (e.g., from PAP air supply 14). A first and second nasal interface may be fluidly coupled to the supply tube via a housing defining a fluid chamber. The first and second nasal interface may each include a generally spherical member that may each have a respective projection configured to be at least partially received within a respective nasal passage of a user. The first and second nasal interface may be independently movable relative to the housing. Breathing apparatus 12 may also include a valve that may be disposed between the fluid chamber and an exhaust passage. The valve may be moveable between an opened position and a closed position. In the closed position, the valve may engage a valve seat, thereby restricting air from being exhausted from the fluid chamber via the exhaust passage. In the open position, the valve may at least partially disengaged the valve seat, thereby allowing air to be exhausted from the fluid chamber via the exhaust passage. The valve seat may include at least one serration extending radially from a valve engagement surface. A diaphragm may be coupled to the valve for moving the valve between the open position and the closed position. A bias chamber may be coupled to the diaphragm for providing a bias force to the diaphragm. A loading fluid impedance may couple the fluid chamber with the bias chamber for regulating the bias force based upon, at least in part, a pressure within the fluid chamber. A venting fluid impedance may couple the fluid chamber with an ambient environment.

With particular reference to FIGS. 2 through 6, breathing apparatus 12 may generally include housing 50. Housing 50 may include one or more supply tubes configured to provide a supply of air. For example, breathing apparatus 12 may include first supply tube 52 and second supply tube 54. Supply tubes 52, 54 may include generally hollow bosses or other openings and/or extensions from housing 50. While not shown, supply first and second supply tubes 52, 54 may each be configured to mate with an additional respective tube that may be coupled (e.g., via a “T” or “Y” fitting, or the like) to supply tube 16 of breathing system 10. As such, supply tubes 52, 54 may provide a supply of air from PAP air supply 14.

Housing 50 may define fluid chamber 56. Supply tubes 52, 54 may be fluidly coupled to fluid chamber 56. Further, breathing apparatus 12 may include first nasal interface 58, and second nasal interface 60. First and second nasal interfaces 58, 60 may be fluidly coupled to supply tubes 52, 54 (and therein fluidly coupled to PAP air supply 14) via fluid chamber 56.

First and second nasal interfaces 58, 60 may each include a generally spherical member (e.g., generally spherical member 62 and generally spherical member 64, respectively). Further, first and second nasal interfaces 58, 60 may each include a respective projection (e.g., projections 66, 68) from generally spherical members 62, 64. Projections 66, 68 may be configured to be at least partially received within a respective nasal passage of a user. Additionally, generally spherical members 62, 64 may each include one or more openings and/or cutouts (e.g., openings 70, 72 in generally spherical member 62, and openings 74, 76 in generally spherical member 64). Openings 70, 72, 74, 76 may be configured to provide fluid communication with supply tubes 52, 54 via housing 50 (e.g., via fluid chamber 56). Accordingly, when projections 66, 68 are at least partially received within a respective nasal passage of a user, the user may be provided with pressurized air from PAP air supply 14.

Generally spherical members 62, 64 may be at least partially received in cooperating recesses of housing 50 (e.g., recess 78 and recess 80, respectively). In one embodiment, recesses 78, 80 may encompass slightly more than half of generally spherical members 62, 64, thereby retaining first and second nasal interfaces 58, 60 to housing 50. For example, recesses 78, 80 may include respective lips 82, 84, which may have an inside diameter that is less than the diameter of generally spherical members 62, 64. Generally spherical members 62, 64 may be assembled to housing 50 (e.g., may be installed in recesses 78, 80) using a snap fit (e.g., resulting from elastic deformation of generally spherical members 62, 64 and/or of lips 82, 84 during assembly), a cap feature (e.g., including lips 82 and/or lips 84) that may be assembled to housing 50 once generally spherical members 62, 64 have been inserted in recesses 78, 80, or similar design feature.

The generally spherical geometry of generally spherical members 62, 64 and the cooperating fit with housing 50 may allow first and second nasal interfaces 58, 60 to pivot and/or rotate within recesses 78, 80. Accordingly, first and second nasal interfaces 58, 60 may be independently movable relative to housing 50. Further, first and second nasal interfaces 58, 60 may also be independently movable relative to one another. The degree of movement of first and second nasal interfaces 58, 60 may depend, at least in part, upon various design features, for example, the relative portion of generally spherical members 63, 64 encompassed by respective recesses 78, 80, the clearance between projections 66, 68 and respective lips 82, 84, etc. Accordingly, the degree of movement of the first and second nasal interfaces 58, 60 may vary depending upon design criteria and user need.

The independent movement of first and second nasal interfaces 58, 60 relative to housing 50 may allow a seal to be maintained between first and second nasal interfaces 58, 60 and a user's respective nasal passages in the event of movement of the user. As described hereinabove, the first and second nasal interfaces 58, 60 may be at least partially received in the nasal passages of the user. As such, housing 50 may generally be disposed beneath the users nose (e.g., resting on the user's upper lip, etc.). While, optionally, head gear (such as an elastic strap or the like) may be used in conjunction with breathing apparatus 12 to locate and/or maintain the position of breathing apparatus 12 relative to the user (e.g., relative to the user's nose), some movement of breathing apparatus 12 relative to the user's head may still occur (e.g., as a result of the user tossing and turning during sleep). The ability of nasal interfaces 58, 60 to move relative to housing 50 may allow the seal and/or positioning of nasal interfaces 58, 60 relative to the user's nasal passages to be maintained. Accordingly, the user may not experience a loss of positive airway pressure. The user may be able to move without dislodging nasal interfaces 58, 60 from the user's nasal passages.

Further, the ability of nasal interfaces 58, 60 to move relative to housing 50 and/or the ability of nasal interfaces 58, 60 to move relative to housing and/or relative to one another may provide some degree of adjustability (e.g., allowing breathing apparatus 12 to fit different users, etc.). For example, movement of nasal interfaces 58, 60 relative to one another and/or relative to housing 50, may allow nasal interfaces 58, 60 to be adjusted to achieve general alignment with the user's nasal passages. As the relative alignment of different user's nasal passages may vary, nasal interfaces may be adjusted (e.g., by movement of nasal interfaces 58, 60 relative to one another and/or relative to housing 50) to accommodate different users. Further, breathing apparatus 12 may include more than one pair of nasal interfaces. The additional pairs of nasal interfaces may include protrusions (e.g., protrusions 66, 68) of different sizes and/or geometries. The different sizes and/or geometries may allow a given user to select a pair of nasal interfaces (e.g., nasal interfaces 58, 60) that best fit the given user's nasal passages.

The degree of movement of housing 50 relative to the user that may be experiences while maintaining the seal between the first and second nasal interfaces 58, 60 and the user's nasal passages may depend, at least in part, upon the freedom of movement between first and second nasal interfaces 58, 60 and housing 50. The freedom of movement between first and second nasal interfaces 58, 60 and housing 50 may depend, at least in part, upon the available movement of first and second nasal interfaces 58, 60 relative to housing 50 (e.g., as discussed above), the relative ease of movement of first and second nasal interfaces 58, 60 within respective recesses 78, 80 (e.g., which may depend, at least in part, upon frictional interactions between first and second nasal interfaces 58, 60 and housing 50), and the like.

Nasal interfaces 58, 60 may be sized, relative to recesses 78, 80, and/or lips 82, 84, to allow facile movement of nasal interfaces 58, 60 relative to housing 50, while minimizing air leakage therebetween. Minimal air leakage and facile movement may be achieved by relatively close tolerances between generally spherical portions 62, 64 and recesses 78, 80, and/or lips 82, 84, in combination with low friction materials. For example, generally spherical portions 62, 64 and lips 82, 84 may each include relatively smooth interacting surfaces (e.g., a high level of surface finish or polish). In addition to relatively smooth interacting surfaces, generally spherical portions 62, 64 and/or the interacting surfaces of recesses 78, 80 (e.g., lips 82, 84) may include low friction materials, such as ultra-high molecular weight polyethylene, fluorinated polyolefins (e.g., tetrafluoroehtylene, such as Teflon™), or the like.

Additionally/alternatively, breathing apparatus 12 may include one or more seals disposed between nasal interfaces 58, 60 and housing 50. For example, breathing apparatus 12 may include first seal 86 disposed between first nasal interface 58 and recess 78. Similarly, breathing apparatus 12 may include second seal 88 disposed between second nasal interface 60 and recess 80. Housing 50 may include one or more features that may at least partially retain first and second seals 86, 88 relative to housing 50. For example, housing 50 may include one or more grooves (e.g., grooves 90, 92) that may accommodate at least a portion of the seals (e.g., first and second seals 86, 88).

A variety of seals may be utilized in the context of breathing apparatus 12. For example, first and second seals 86, 88 may include a brush seal, a felt ring or an o-ring (e.g., which may include a relatively lubricious material such as a polyolefin, fluorinated polyolefin, a low friction elastomer, or the like). In addition to reducing air leakage between housing 50 and first and second nasal interfaces 58, 60, while allowing facile movement of first and second nasal interfaces 58, 60 relative to housing 50, first and second seals 86, 88 may also facilitate assembly of breathing apparatus 12. For example, in an embodiment in which first and second nasal interfaces 58, 60 may be snap-fit into recesses 78, 80, recesses 78, 80 may have a diameter (e.g., at lips 82, 84) that may be larger than the diameter of generally spherical portions 62, 64. The inside diameter of seals 86, 88 may be less than the diameter of generally spherical portions 62, 64, thereby allowing first and second nasal interfaces 58, 60 to be retained to housing 50. Seals 86, 88 may include a relatively compliant and/or elastically deformable material, which may elastically deform to allow the snap-fit insertion of first and second nasal interfaces 58, 60 into recesses 78, 80. Subsequent to snap-fit insertion of first and second nasal interfaces 58, 60 into recesses 78, 80, first and second seals 86, 88 may elastically recover to an inside diameter that is less than the diameter of generally spherical portions 62, 64, thereby retaining first and second nasal interfaces 58, 60 to housing 50.

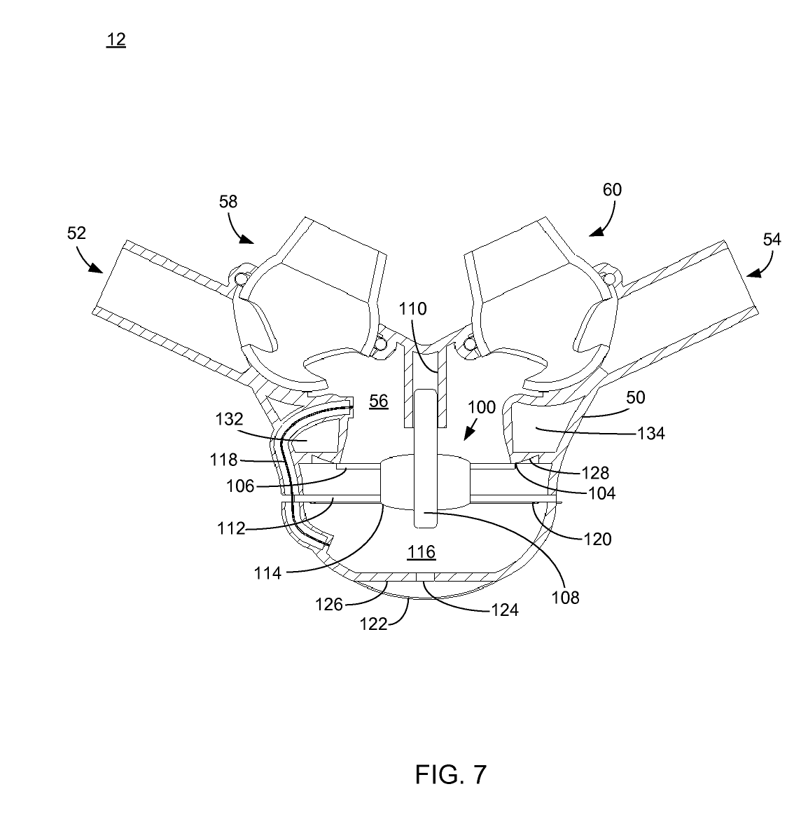

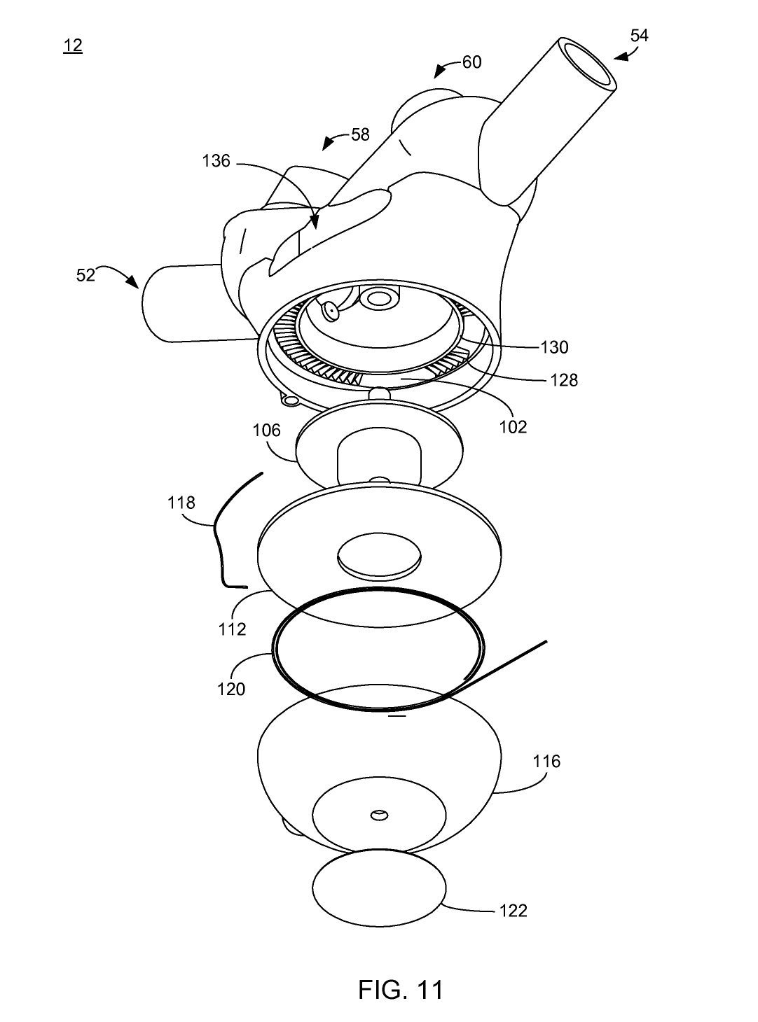

As discussed above, and referring also to FIGS. 7 through 12, breathing apparatus 12 may include a regulator that may reduce exhalation resistance experienced by a user, e.g., by facilitating the exhaust of an exhaled breath from breathing apparatus 12. As discussed above, breathing apparatus 12 may include a valve (e.g., valve 100) disposed between fluid chamber 56 and an exhaust passage 102 (shown in FIG. 11). Valve 100 may be movable between an opened position and a closed position. In the closed position valve 100 may engage a valve seat (e.g., valve seat 104), thereby restricting air from being exhausted from fluid chamber 56. In the opened position valve 100 may be at least partially disengaged from valve seat 104, thereby allowing air to be exhausted from fluid chamber 56 via exhaust passage 102. As mentioned above, exhaust passage 102 may allow for relatively low resistance exhaust of exhaled air from breathing apparatus 12. As such, the regulator may, at least in part, reduce exhalation resistance experienced by the user, and may also allow for the use of a relatively small diameter supply tube (e.g., supply tube 106), as exhaled air need not be exhausted via the supply tube or a dedicated exhaust tube (e.g., which may typically exhaust at PAP air supply 14).

The regulator (including valve 100 selectively engaging valve seat 104) may be a pressure biased regulator such that valve 100 may open at pressures above the average supply pressure of the pressurized air supplied by PAP air supply 14. Accordingly, valve 100 may remain in the closed position during the inhalation cycle, during which air is supplied from PAP air supply 14. As such, pressurized air supplied from PAP air supply 14 may be directed into the user's air pathways via fluid chamber 56, first and second nasal interfaces 58, 60 and the user's nasal passages. However, valve 100 may move to the open position during the exhalation cycle, during which the user may exhale and the pressure within fluid chamber 56 may rise above the average supply pressure. The opening of valve 100 during the exhalation cycle 100 may reduce the exhalation resistance experienced by the user, which may, thereby, reduce discomfort experienced by the user.

Valve 100 may generally include valve plate 106 which may engage valve seat 104. Valve plate 106 may include a generally rigid member (e.g., formed of a suitable plastic or metal) that may generally translate as a unit to move between the opened and the closed position, rather than deforming away from valve seat 104. Valve plate 106 may be coupled to valve shaft 108. At least a portion of valve shaft 108 may be disposed within a guide passage, such as guide boss 110. Guide boss 110 may allow valve plate 106 (along with valve shaft 108) to translate in a generally axial manner thereby maintaining the general positional orientation of valve plate 106 relative to valve seat 104.

Valve 100 may be coupled to a diaphragm (e.g., diaphragm 112) for moving valve 100 between the opened and the closed position. As shown, valve plate 106 and diaphragm 112 may be coupled to one another via valve member 114, which may be disposed on valve shaft 108. Valve member 114 may include a generally cylindrical member (e.g., of plastic, metal, or the like), which may be coupled to each of valve plate 106 and diaphragm 112, as well as to valve shaft 108. Valve member 114 may be coupled to valve plate 106, valve shaft 108 and diaphragm 112 by any suitable means (including a different means for each coupling), including, but not limited to, an adhesive, mechanical fastener, welding (e.g., thermal welding, ultrasonic welding, friction welding, etc.), a friction fit (e.g., a press fit), or other suitable means. Accordingly, valve plate 106, valve shaft 108, and valve member 114 may generally translate in response to a deflection of diaphragm 112.

Diaphragm 112 may be coupled to a bias chamber (e.g., bias chamber 116), which may provide a bias force to the diaphragm. The bias force provided by bias chamber 116 may include pressurized fluid (e.g., pressurized air, in the case of breathing apparatus 12) contained within bias chamber 116. The pressurized air contained within bias chamber 116 may exert a bias force on diaphragm 112. The bias force exerted on diaphragm 112 may be transferred to valve plate 106 via valve member 114, thereby providing a closing force urging valve plate 106 against valve seat 104. When the user exhales, the pressure of the exhaled air received within fluid chamber 56 may urge valve plate 106 toward the open position (e.g., as a result of the pressure acting on valve plate 106). When the pressure acting on valve plate 106 exceeds the bias force on diaphragm 112, diaphragm 112 may deflect at least partially towards bias chamber 116. The at least partial deflection of diaphragm 112 towards bias chamber 116 may allow valve plate 106 to move to the open position, thereby allowing the exhaled air within fluid chamber 56 to be vented via exhaust passage 102.

Diaphragm 112 may include a resiliently deformable member, e.g., allowing diaphragm 112 to deflect at least partially towards bias chamber 116 when the force exerted on valve plate 106 exceeds the force exerted on diaphragm 112 by the pressurized fluid within bias chamber 116. For example, diaphragm 112 may be formed of an elastomeric membrane, or other suitable resiliently deformable material. Further, as described above, valve plate 106 may move to the opened position when the force exerted on valve plate 106 (e.g., by exhaled air within fluid chamber 56) exceeds the pressure exerted on diaphragm 112 by the pressurized fluid within bias chamber 116. The force urging valve plate 106 towards the open position may be, at least in part, a function of the pressure of the exhaled air within fluid chamber 56 multiplied by the surface area of valve plate 106 witnessing the pressure of the exhaled air within fluid chamber 56. Similarly, the bias force exerted on diaphragm 112 may be, at least in part, a function of the pressure of the fluid within bias chamber 116 multiplied by the surface are of diaphragm 112 witnessing the pressure of the fluid within bias chamber 116. Accordingly, an opening force of the valve may be based upon, at least in part, a ratio of the surface area of valve plate 106 and the surface area of diaphragm 112.

The regulator, including valve 100, may include a loading fluid impedance that may couple the fluid chamber with the bias chamber for regulating the bias force based upon, at least in part, a pressure within the fluid chamber. As described above, the bias force exerted on diaphragm 112 may be, at least in part, a function of the pressure of the pressurized fluid within bias chamber 116. In some embodiments, it may be desirable that the pressure of exhaled air required to open valve 100 (e.g., to move valve plate 106 to the opened position) may be slightly greater than the average pressure of the air supplied to the user.

The loading fluid impedance may include a fluid passage having an associated loading impedance pressure drop. The loading impedance pressure drop may impart a hysteresis on the bias chamber 116, such that pressure within bias chamber 116 may not immediately vary with changes in pressure in fluid chamber 56. Accordingly, when the pressure within fluid chamber 56 is greater than the pressure within bias chamber 116, the pressure within bias chamber 116 may rise over time to the pressure within fluid chamber 56. Similarly, when the pressure within bias chamber 116 is greater than the pressure within fluid chamber 56, the pressure within bias chamber 116 may decrease over time to the pressure within fluid chamber 56. However, due to the loading impedance pressure drop, the pressure within bias chamber 116 may not instantly change to match the pressure within fluid chamber 56. As such, the pressure within bias chamber 116 may approach the general average pressure within fluid chamber 56 (e.g., an average of the supply air pressure during inhalation, the exhalation air pressure and a low pressure condition between inhalation and exhalation). Additionally, the pressure within bias chamber 116 may vary over time in the even that the average pressure within fluid chamber 56 varies over time.

According to one embodiment, the loading fluid impedance fluid passage having an associated loading impedance pressure drop may include a small diameter tube (e.g., supply capillary tube 118). For example, supply capillary tube 118 may have in inside diameter of about 0.1 mm and a length of about 48 mm. Additionally/alternatively the fluid passage having an associated loading impedance pressure drop may include, for example, a small diameter orifice, a semi-permeable plug or membrane, as well as various additional structures that may impart the desired pressure drop coupling fluid chamber 56 and bias chamber 116. In various embodiments, the loading fluid impedance may include an associated filter (e.g., which may include a hydrophobic filter) that may reduce the likelihood of loading fluid impedance becoming obstructed (e.g., by a foreign material, water, or the like).

The breathing apparatus may further include an initial loading valve selectively fluidly coupling the fluid chamber and the bias chamber. For example, while not shown, breathing apparatus 12 may include a manually and/or automatically actuable loading valve that may fluidly couple fluid chamber 56 and bias chamber 116. For example, in the case of a manually actuable loading valve, during initial operation of breathing apparatus 12, the user may actuate the loading valve to fluidly couple fluid chamber and bias chamber 116 via a relatively low impedance fluid pathway. When the loading valve is actuated, the pressure within bias chamber 116 may rapidly rise to the pressure within fluid chamber 56. Accordingly, the loading valve may allow bias chamber 116 to achieve a pressure that may generally be the average pressure within fluid chamber 56. As such, the initial settling time for the pressure within bias chamber 116 may be decreased relative to the settling time that may occur when bias chamber 116 is charged via the loading fluid impedance.

The regulator may further include a venting fluid impedance coupling the bias chamber (e.g., bias chamber 116) with second pressure source. In one embodiment, the second pressure source may include a pressure lower than the average pressure within fluid chamber 56. For example, the second pressure source may be an ambient environment (e.g., an ambient environment outside of breathing apparatus 12). The venting fluid impedance coupling bias chamber with the ambient environment may allow the continual and gradual release of pressure from bias chamber 116. The continual and gradual release of pressure from bias chamber 116 may prevent the continual accumulation of pressure within bias chamber 116. For example, the venting fluid impedance may assist in maintaining a constant pressure within bias chamber 116 even as diaphragm 112 moves during opening and closing of valve 100 (e.g., the opening and closing of valve plate 106 relative to valve seat 104).

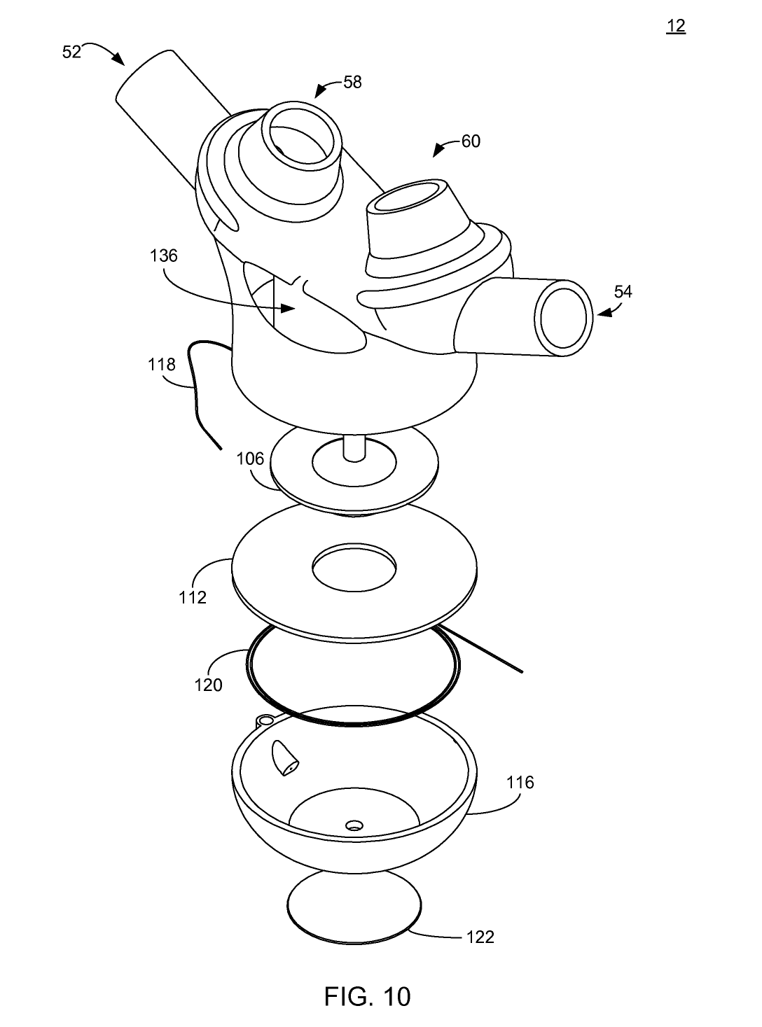

Similar to the loading fluid impedance, the venting fluid impedance may include a fluid passage having an associated venting impedance pressure drop. In one embodiment, the fluid passage having an associated venting impedance pressure drop may include a small diameter tube (e.g., venting capillary tube 120, best shown in FIGS. 10 through 12). For example, venting capillary tube 120 may have in inside diameter of about 0.1 mm and a length of about 216 mm. Additionally/alternatively the fluid passage having an associated venting impedance pressure drop may include, for example, a small diameter orifice, a semi-permeable plug or membrane, as well as various additional structures that may impart the desired pressure drop coupling bias chamber 116 to the second pressure source (e.g., the ambient environment). In various embodiments, the venting fluid impedance may include an associated filter (e.g., which may include a hydrophobic filter) that may reduce the likelihood of loading fluid impedance becoming obstructed (e.g., by a foreign material, water, or the like).

The venting impedance pressure drop may be greater than a loading impedance pressure drop associated with the loading fluid impedance. As such, the pressure within bias chamber 116 may generally more closely approximate the average pressure within fluid chamber 56 rather than the pressure of the second pressure source. Consistent with the foregoing example, the venting impedance pressure drop may be greater than the loading impedance pressure drop as a result of the greater length of venting capillary tube 120 compared to supply capillary tube 118. However, other techniques may equally be utilized depending upon the structure of the venting fluid impedance and the loading fluid impedance.

The regulator may further include an expandable member coupled to the bias chamber. As the volume within bias chamber 116 may be relatively small, the deflection of diaphragm 112 into bias chamber 116 during the opening of valve plate 106 may result in a relatively significant increase in the pressure within bias chamber 116. The relatively significant increase in the pressure within bias chamber 116 may result in an increase in the bias force countering the opening of valve plate 106. The increase in the bias force may impede the full opening of valve plate 106, which may result in an increase in the exhalation resistance experienced by the user. The expandable member may include resilient cap 122, which may be fluidly coupled to bias chamber 116 by way of a fluid passage (e.g., opening 124) in wall 126 defining at least a portion of bias chamber 116. During opening of valve plate 106 (e.g., as a result of the user exhaling), diaphragm 112 may at least partially deflect into bias chamber 116, resulting in a decrease in the volume of bias chamber 116, and an attendant increase in the pressure within bias chamber 116. The increase in the pressure within bias chamber 116 may cause resilient cap 122 to expand outwardly from bias chamber 116. The outward expansion of resilient cap 122 may provide an increase in the effective volume of bias chamber 116, thereby decreasing the pressure within bias chamber 116. Accordingly, resilient cap 122 may attenuate the increase in pressure within bias chamber 116 (e.g., by maintaining a generally constant effective volume of bias chamber 116) during the opening of valve plate 106, and may allow valve plate 106 to fully open.

Resilient cap 122 may include any suitable resiliently expandable material and/or structure. For example, resilient cap 122 may include an elastomeric membrane or structure. Various additional/alternative configurations may similarly be utilized. For example, the expandable member may include a spring loaded piston that may increase in volume in response to an increase in pressure, as well as various other suitable configuration. Additionally, while resilient cap 122 has been shown fluidly coupled to bias chamber 116 by way of opening 124, the fluid passage coupling resilient cap 122 and bias chamber 116 may additionally/alternatively include a flow control means (e.g., a controlled diameter orifice, a controlled flow porous structure, etc.), which may at least partially dampen the flow of fluid from bias chamber 116. At least partially dampening the flow of fluid from bias chamber 116 may control and/or reduce the oscillation of valve plate 106, which may result from uncontrolled outward expansion and subsequent recovery of resilient cap 122.

While the regulator described herein above has been discussed in the context of an exhaust regulator for a breathing apparatus, it should be understood that broader applicability may be realized. Generally, the regulator may be employed in any application utilizing a valve actuated based upon, at least in part, an applied pressure.

In addition to reducing exhalation resistance experienced by the user, breathing apparatus 12 may also incorporate features that may reduce noise associated with exhausting exhaled air from breathing apparatus. As discussed above, valve 100 (e.g., include valve plate 106 that may selectively engage/disengage valve seat 104) may be selectively opened (e.g., in response to a user exhaling) to provide an exhaust pathway from breathing apparatus 12. In one embodiment, valve seat 104 may be configured to reduce noise associated with the passage of air through valve 100. Valve seat 104 may include at least one serration (e.g., serration 128) extending radially from a valve engagement surface (e.g., valve engagement surface 130). In one embodiment, the depth of serration 128 may increase radially away from the valve engagement surface. However, in other embodiments, the serration may have a generally uniform depth extending radially from the valve engagement surface. More particularly, and as shown in, e.g., FIG. 11, breathing apparatus 12 may include a plurality of serrations disposed about the circumference of valve engagement surface 130. The serrations (e.g., serration 128) disposed around the circumference of engagement surface 130 of valve seat 104 may reduce the occurrence of air flow over the edge of valve seat 104 (e.g., air flowing between valve plate 106 and valve seat 104) causing valve plate 106 to vibrate, especially during the initial opening of valve 100.

According to another aspect, the exhaust pathway may include an exhaust passage that may be configured to redirect exhaust air exiting the valve in a first direction to a substantially different second direction. For example, and with particular reference to FIGS. 7, 10, and 11, breathing apparatus 12 may include exhaust port 102 through which exhaled air may exit from fluid chamber 56 when valve 100 is in the opened position (e.g., when valve plate 106 is disengaged from valve seat 104). The exhaust air exiting via exhaust port 102 (located proximate a first side of housing 50) may be directed through one or more exhaust passages (e.g., exhaust passages 132, 134), and may exit housing 50 via exhaust vent 136, which may be disposed on a second side of housing 50, which is generally opposed to the first side of the housing including exhaust port 102. The tortuous exhaust path provided by exhaust passages 132, 134 may reduce the noise associated with the exhaled air leaving breathing apparatus 12.

Additionally, the exhaust passage may include a textured interior surface. For example, the interior surfaces of exhaust passages 132, 134 may include a textured surface finish. The textured surface finish of exhaust passages 132, 134 may at least somewhat reduce the transmission of sound via exhaust passages 132, 134. As such, the sound exiting breathing apparatus 12 may be reduced.

As discussed above, breathing apparatus 12 may include a regulator that may reduce exhalation resistance experienced by a user, e.g., by facilitating the exhaust of an exhaled breath from breathing apparatus 12. As also described above, in a generally manner, the regulator of breathing apparatus 12 may generally include a valve disposed between a fluid chamber (e.g., fluid chamber 56, described above) and the second fluid passage (e.g., exhaust passage 102, also described above). The valve may be moveable between an open position, allowing fluid communication between the fluid chamber and the exhaust passage, and a closed position, restricting fluid communication between the fluid chamber and the exhaust passage.

The regulator, including the valve (e.g., valve 100), may be a pressure biased regulator, such that the valve may open at pressures above a threshold pressure. In this regard, the valve may be coupled to a diaphragm (e.g., diaphragm 112) for moving the valve between the opened and the closed positions. As described above, diaphragm 112 may be coupled to a bias chamber (e.g., bias chamber 116), which may provide a bias force to the diaphragm. The bias force provided by bias chamber 116 may include pressurized fluid (e.g., pressurized air, in the case of breathing apparatus 12) contained within bias chamber 116. The pressurized air contained within bias chamber 116 may exert a bias force on diaphragm 112. The bias force exerted on diaphragm 112 may be transferred to the valve, thereby providing a closing force urging the valve towards the closed position. When the user exhales, the pressure of the exhaled air received within fluid chamber 56 may urge the valve toward the open position (e.g., as a result of the pressure acting on the valve). When the pressure acting on the valve exceeds the bias force on diaphragm 112, diaphragm 112 may deflect at least partially towards bias chamber 116. The at least partial deflection of diaphragm 112 towards bias chamber 116 may allow the valve to move to the open position, thereby allowing the exhaled air within fluid chamber 56 to be vented via exhaust passage 102.

The bias chamber 116 may be provided with the pressurized fluid (e.g., which may provide the bias force) via a loading fluid passage, which may fluidly couple bias chamber 116 with a loading fluid source for regulating the bias force. As described above, the loading fluid passage may include a loading fluid impedance (e.g., supply capillary tube 118) fluidly coupling fluid chamber 56 and the bias chamber 116 Accordingly, pressurized air within fluid chamber 56 may provide, at least in part, the loading fluid source. As such, the bias force provided may be based upon, at least in part, a pressure within fluid chamber 56. The bias force provided by diaphragm 112 may be based upon, at least in part, a loading impedance pressure drop associated with loading fluid impedance (i.e., supply capillary tube 118 in the foregoing example).

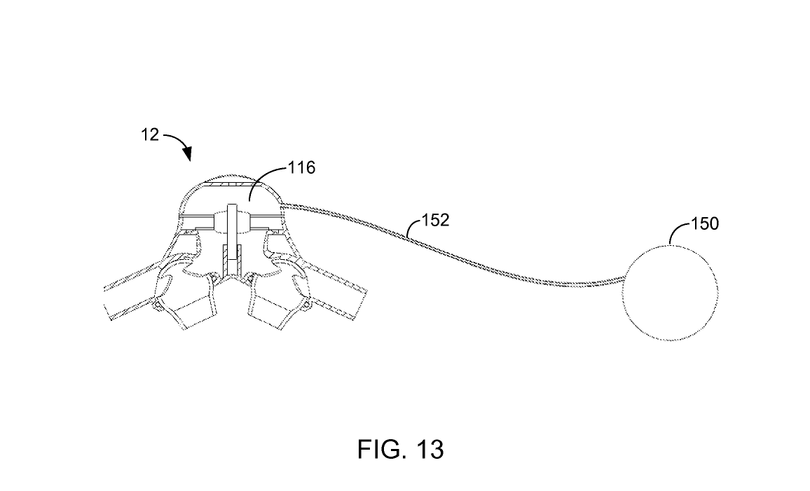

According to a further embodiment, bias chamber 116 may be coupled to a loading fluid source other than fluid chamber 56. In one such embodiment, the loading fluid source may include a voice coil driven source of pressurized fluid. For example, and referring also to FIG. 13, voice coil driver 150 may include an at least partially sealed voice coil that may be fluidly coupled to bias chamber 116 via loading tube 152 (e.g., which may be a separate lumen included within supply tube 16 and/or may be a separate tube). Voice coil driver 150 may be integrated into PAP air supply 14 and/or may be a separate component. The current supplied to voice coil driver 150 may control the pressure supplied to bias chamber 116. For example, the current supplied to voice coil driver 150 may control the displacement of the voice coil, and therein control the volume of fluid (e.g., air) transferred to bias chamber 116, and the resulting pressure within bias chamber 116. Coupling voice coil driver 150 to bias chamber 116 via loading tube 152 may allow voice coil driver 150 to be remotely located, thereby reducing the size and weight of breathing apparatus 12. In one particular example, loading tube 152 may include an approximately one meter long tube having a two millimeter inside diameter. In such an embodiment, the loading system (including voice coil 150 coupled to bias chamber 116 via loading tube 152) may have a frequency response of about 20 Hz, which may allow for very rapid control of the opening pressure of the valve.

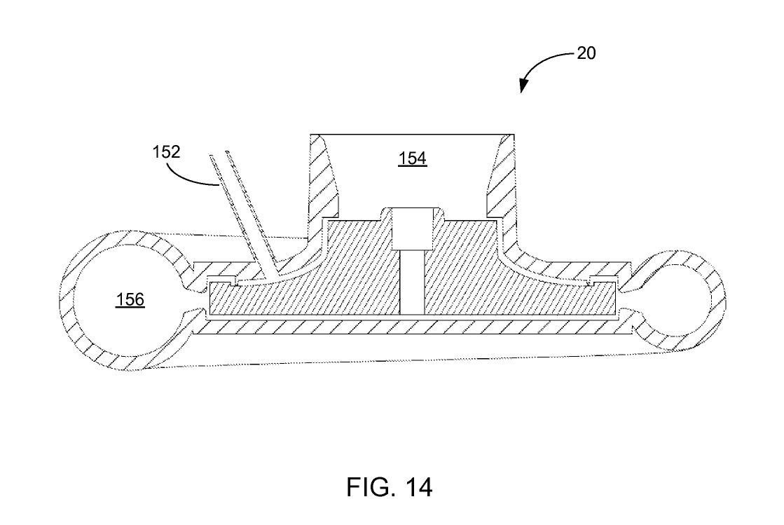

In a further embodiment, the loading fluid source may include a blower (e.g., blower 20 of PAP air supply 14). For example, the loading fluid passage may be coupled to the output of blower 20. The pressure provided to bias chamber 116 may be regulated using a valve, flow restriction, supply tube having a predetermined pressure drop, or the like. In a further embodiment, the loading fluid passage may be fluidly coupled to the blower between a blower inlet and a blower outlet. For example, and referring also to FIG. 14, the loading fluid passage (e.g., loading tube 152) may be coupled to blower between blower inlet 154 and blower outlet 156. The pressure of the loading fluid source (e.g., and therein the pressure supplied to bias chamber 116 via loading tube 152) may be based upon, at least in part, the location of loading tube 152 between blower inlet 154 and blower outlet 156. Accordingly, loading tube 152 may be located between blower inlet 154 and blower outlet 156 to provide a bias pressure that is a desired proportion of the outlet pressure of blower 20.

Additionally, while loading tube 152 is illustrated in FIG. 14 as being flush and generally normal with the interior housing of blower 20, other embodiments may be utilized to vary the loading fluid pressure provided by blower 20. For example, loading tube 152 may project into the interior housing of blower 20 and may be angled relative to a flow path of air within blower 20 (e.g., in the manner of a pitot tube), thereby altering the pressure supplied to bias chamber 116 via loading tube 152 as a result of the dynamic pressure witnessed by loading tube 152.

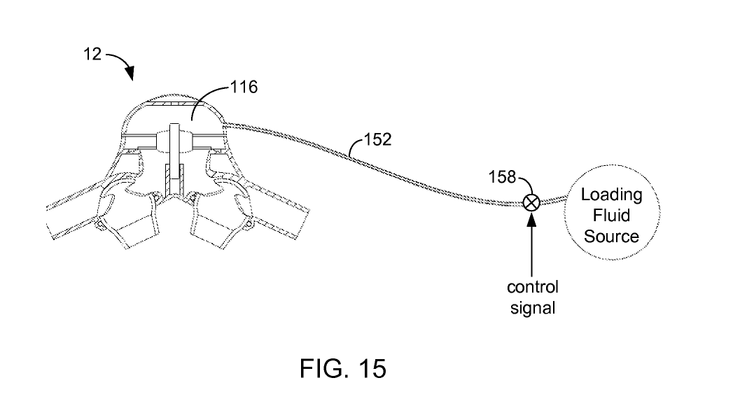

Consistent with any of the preceding embodiments, a valve may couple the loading fluid source and the bias chamber. The valve may be configured to regulate the bias force by regulating a pressure within the bias chamber. For example, and referring also to FIG. 15, the loading fluid source may be coupled to bias chamber 116 of breathing apparatus 12 via loading tube 152. Valve 158 may be coupled between the loading fluid source and bias chamber 116 of breathing apparatus 12. While valve 158 is shown associated with loading tube 152, it should be appreciated that valve 158 may similarly be associated with either breathing apparatus 12 and/or the loading fluid source. Valve 158 may include any suitable valve, such as a solenoid valve, which may be selectively opened and closed to allow pressurized fluid to flow between the loading fluid source and bias chamber 116, and/or to control a pressure drop therebetween. The opening and closing of valve 158 may be controlled by, e.g., a control signal from controller 18 of PAP air supply 14. In some embodiments, valve 158 may include a pulse width modulated duty cycle (e.g., based upon, at least in part, a pulse width modulated control signal) to regulate the pressure within bias chamber 116. For example, the pulse width modulated control signal may provide a desired duty cycle based upon a pressure of the loading fluid source and a venting or bleed rate of pressurized fluid from bias chamber 116 (e.g., via venting capillary tube 120, discussed above).

While the regulator may include a venting fluid impedance coupling the bias chamber with second pressure source (e.g., which may include, but is not limited to, an ambient environment), as described above, in various embodiments (e.g., in which the loading fluid source may prevent and/or reduce the continual accumulation of pressure within bias chamber 116), the venting fluid impedance may not be necessary. In such embodiments, the regulator may not include a venting fluid impedance. Similarly, while the regulator may also include an expandable member coupled to the bias chamber, in which the expandable member may be configured to expand in response to an increase in a bias chamber pressure associated with a deflection of the diaphragm, in various embodiments (e.g., in which the bias pressure may be regulator so as to prevent or reduce an increase in bias chamber pressure), the expandable member may not be necessary. In such embodiments, the expandable member need not be included.

The valve may include a valve member configured to engage a valve seat in the closed position and may be configured to at least partially disengage the valve seat in the open position. As described above, according to an embodiment, the valve member may include a valve plate (e.g., valve plate 106) that may at least partially engage and disengage valve seat 104 to open and close exhaust passageway 102.

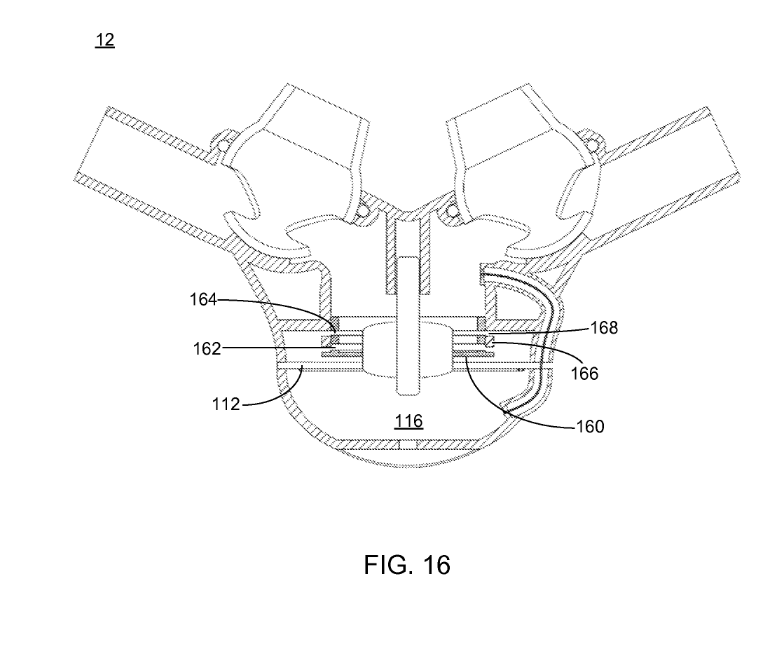

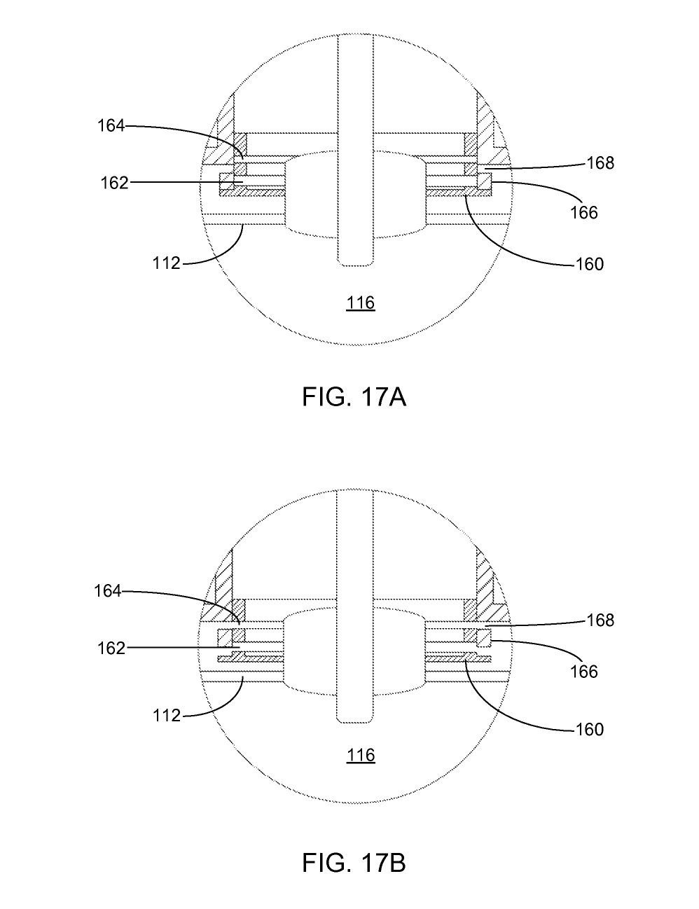

According to a further embodiment, the valve may be configured as a slot valve. In an exemplary embodiment of a slot valve, the valve member may include a valve body having at least one radial slot. The at least one radial slot may be at least partially obstructed by the valve seat in the closed position. For example, and referring also to FIGS. 16 through 17B, valve body 160 may include a generally cylindrical body that may include a first generally radial slot 162 and a second generally radial slot 164. First radial slot 162 may be at least partially axially spaced from the second radial slot 164 on valve body 160.

In a similar manner as described above with respect to valve plate 106, valve body 160 may generally axially translate in response to deflection of diaphragm 112 when a force exerted on valve body 160 (e.g., resulting from an exhalation pressure) exceeds the bias force exerted on diaphragm 112 by the pressure of the loading fluid within bias chamber 116. With particular reference to FIG. 17A, when the force exerted on valve body 160 is less than the force exerted on diaphragm 112, valve body 160 may be in a closed position. In the closed position, shown in FIG. 17A, first slot 162 and second slot 164 may be at least partially obstructed by a valve seat (e.g., in particular by respective valve seat members 166, 168, defining at least one opening therebetween).

When the force on valve body 160 exceeds the force exerted on diaphragm 112 (e.g., during exhalation of a user of breathing apparatus 12), valve body 160 may translate generally axially towards an open position. In the open position, shown in FIG. 17B, first slot 162 and second slot 164 may at least partially align with the at least one opening defined between valve seat members 166, 168. The at least partial alignment of first slot 162 and second slot 164 with the opening(s) defined between valve seat members 166, 168 may allow exhales air to flow from fluid chamber 56 through exhaust passageway 102.

According to one aspect, the slot valve arrangement, shown in FIGS. 16, 17A, and 17B may allow for a greater open flow area between fluid chamber 56 and exhaust passageway 102 for a given axial displacement of diaphragm 112 (assuming a similar valve diameter), as compared to valve plate 106. For example, for a given axial displacement of diaphragm 112, the opening provided by valve plate 106 may be generally comparable to the opening provided by slot 164 relative to valve seat member 166. In addition to this opening, the slot valve may provide the additional open flow area defined by second radial slot 164 and the opening between valve seat members 166, 168.

A number of implementations have been described. Nevertheless, it will be understood that various modifications may be made. Accordingly, other implementations are within the scope of the following claims.

What is claimed is:

1. A breathing apparatus comprising:
a first supply tube configured to provide a supply of air;
a first and second nasal interface fluidly coupled to the first supply tube via a housing, the first and second nasal interface each including a generally spherical member having a respective projection configured to be at least partially received within a respective nasal passage of a user, the first and second nasal interface being independently movable relative to the housing.

2. The breathing apparatus according to claim 1, further comprising a second supply tube configured to provide a supply of air, the second supply tube fluidly coupled to the first and second nasal interface via the housing.

3. The breathing apparatus according to claim 1, wherein the generally spherical member of each of the first and second nasal interface includes an opening configured to provide fluid communication via the housing.

4. The breathing apparatus according to claim 1, further comprising a first seal disposed between the first nasal interface and the housing, and a second seal disposed between the second nasal interface and the housing.

5. The breathing apparatus according to claim 4, wherein each of the first seal and the second seal include a brush seal.

6. The breathing apparatus according to claim 4, wherein each of the first seal and the second seal include a felt ring.

7. The breathing apparatus according to claim 4, wherein each of the first seal and the second seal include an o-ring.

8. A breathing apparatus comprising:
a supply tube configured to provide a supply of air;
a first and second nasal interface fluidly coupled to the supply tube via a housing defining a fluid chamber, the first and second nasal interface each including a generally spherical member having a respective projection configured to be at least partially received within a respective nasal passage of a user, the first and second nasal interface being independently movable relative to the housing;
a valve disposed between the fluid chamber and an exhaust passage, the valve moveable between a closed position, being engaged with a valve seat, restricting air from being exhausted from the fluid chamber via the exhaust passage, and an open position, being at least partially disengaged with the valve seat, allowing air to be exhausted from the fluid chamber via the exhaust passage, the valve seat including at least one serration extending radially from a valve engagement surface;
a diaphragm coupled to the valve for moving the valve between the open position and the closed position;
a bias chamber coupled to the diaphragm for providing a bias force to the diaphragm;
a loading fluid impedance coupling the fluid chamber with the bias chamber for regulating the bias force based upon, at least in part, a pressure within the fluid chamber; and
a venting fluid impedance coupling the fluid chamber with an ambient environment.

9. The breathing apparatus according to claim 8, further comprising a first seal disposed between the first nasal interface and the housing, and a second seal disposed between the second nasal interface and the housing.

10. The breathing apparatus according to claim 8, wherein the at least one serration includes a plurality of serrations disposed about the circumference of the valve engagement surface.

11. The breathing apparatus according to claim 8, wherein the exhaust passage is configured to redirect exhaust air exiting the via the valve in a first direction to a substantially different second direction.

12. The breathing apparatus according to claim 11, wherein the exhaust passage is configured to redirect exhaust air proximate a first side of the housing to a second side of the housing generally opposed to the first side of the housing.

13. The breathing apparatus according to claim 8, wherein the loading fluid impedance includes a fluid passage having an associated loading impedance pressure drop, and wherein the venting fluid impedance includes a fluid passage having an associated venting impedance pressure drop.

14. The breathing apparatus according to claim 13, wherein the venting impedance pressure drop is greater than the loading impedance pressure drop.

15. The breathing apparatus according to claim 8, further including an expandable member coupled to the bias chamber, the expandable member configured to expand in response to an increase in a bias chamber pressure associated with a deflection of the diaphragm.

16. The breathing apparatus according to claim 8, further comprising an initial loading valve selectively fluidly coupling the fluid chamber and the bias chamber.

17. The breathing apparatus according to claim 8, wherein an opening force of the valve is based upon, at least in part, a ratio of a valve surface area and a diaphragm surface area.

18. The breathing apparatus according to claim 8, wherein the at least one serration has a depth increasing radially away from the valve engagement surface

19. A breathing apparatus comprising:
a supply tube configured to provide a supply of air;
a first and second nasal interface fluidly coupled to the supply tube via a housing defining a fluid chamber, the first and second nasal interface each including a generally spherical member having a respective projection configured to be at least partially received within a respective nasal passage of a user, the first and second nasal interface being independently movable relative to the housing;
a valve disposed between the fluid chamber and an exhaust passage, the valve moveable between a closed position restricting air from being exhausted from the fluid chamber via the exhaust passage, and an open position allowing air to be exhausted from the fluid chamber via the exhaust passage;
a diaphragm coupled to the valve for moving the valve between the open position and the closed position;
a bias chamber coupled to the diaphragm for providing a bias force to the diaphragm;
a loading fluid passage fluidly coupling the bias chamber with a loading fluid source for regulating the bias force; and
a venting fluid impedance coupling the fluid chamber with an ambient environment.

20. The breathing apparatus according to claim 19, wherein the loading fluid passage includes a loading fluid impedance having an associated loading impedance pressure drop, the loading fluid impedance fluidly coupling the fluid chamber and the bias chamber for regulating the bias force based upon, at least in part, a pressure within the fluid chamber.

21. The breathing apparatus according to claim 19, wherein the loading fluid source includes a voice coil driven source of pressurized fluid.

22. The breathing apparatus according to claim 19, wherein the loading fluid source includes a blower, and the loading fluid passage fluidly couples the blower and the bias chamber.

23. The breathing apparatus according to claim 19, further including a valve coupling the loading fluid source and the bias chamber, the valve configured to provide a pulse width modulated duty cycle to regulate the bias force by regulating a pressure within the bias chamber.

24. The breathing apparatus according to claim 19, further including an expandable member coupled to the bias chamber, the expandable member configured to expand in response to an increase in a bias chamber pressure associated with a deflection of the diaphragm.

25. The breathing apparatus according to claim 19, wherein the valve includes a valve member configured to engage a valve seat in the closed position and configured to at least partially disengage the valve seat in the open position.

26. The breathing apparatus according to claim 25, wherein the valve member includes a valve plate.

27. The breathing apparatus according to claim 25, wherein the valve member includes a valve body having at least a first radial slot and a second radial slot, the first radial slot being at least partially axially spaced from the second radial slot, the first radial slot and the second radial slot being at least partially obstructed by the valve seat in the closed position.
